(19) United States
(12) Patent Application Publication
(10) Pub. No.: US 2013/0217940 A1
(43) Pub. Date: Aug. 22, 2013

(54) Alkylation of Benzene and/or Toluene with Methanol

(71) Applicants: Xiaobo Zheng, Houston, TX (US); John Di-Yi Ou, Houston, TX (US); Mark P. Hagemeister, Houston, TX (US)

(72) Inventors: Xiaobo Zheng, Houston, TX (US); John Di-Yi Ou, Houston, TX (US); Mark P. Hagemeister, Houston, TX (US)

(21) Appl. No.: 13/483,836

(22) Filed: May 30, 2012

Related U.S. Application Data
(60) Provisional application No. 61/506,309, filed on Jul. 11, 2011.

(51) Int. Cl. C07C 2/86

(52) U.S. Cl. CPC C07C 2/864
USPC 585/469

(57) ABSTRACT

The present inventors have surprisingly discovered that paraxylene selectivity is found to increase as the amount of coke on catalyst increases. In embodiments the paraxylene selectivity and productivity is maximized by controlling the amount of coke on the catalyst while maintaining xylene yield at an acceptable value. The control of coke may be achieved by one or a combination of the following techniques: increasing catalyst on-oil time, decreasing catalyst residence time in the regenerator, reducing the air or oxygen supply to the regenerator, and decreasing catalyst circulation rate, or a combination thereof.

### PRIORITY CLAIM

This application claims the benefit of Provisional Application No. 61/506,309, filed Jul. 11, 2011, the disclosure of which is incorporated by reference in its entirety.

### FIELD OF THE INVENTION

The invention relates to the improvement in the alkylation of aromatics using a fluidized bed reactor, and is more particularly related to improving selectivity to paraxylene in the alkylation of benzene and/or toluene.

### BACKGROUND OF THE INVENTION

It is well-known to manufacture xylenes by the alkylation of toluene and/or benzene with methanol, and in particular to selectively make paraxylene product using zeolite catalyst. See, for instance, U.S. Pat. Nos. 4,002,698; 4,356,338; 4,423,266; 5,675,047; 5,804,690; 5,939,597; 6,028,238; 6,046,372; 6,048,816; 6,156,949; 6,423,879; 6,504,072; 6,506,954; 6,538,167; and 6,642,426. Paraxylene selectivity is highly sought after because of the economic importance of paraxylene relative to meta- and orthoxylene. Although each of the xylene isomers have important and well-known end uses, paraxylene is currently the most economically valuable, serving as an intermediate in such important and diverse end uses as bottle plastic and polyester fibers.

One of the problems in the alkylation of toluene and/or benzene with methanol using zeolite catalysts is that the zeolite catalyst gradually loses its activity as coke accumulates on it. Typically after a period of time in contact with the reactants, referred to as the “on-oil” time, the catalyst is regenerated. Catalyst regeneration involves, at least in part, burning off most if not all of the coke, typically with an oxygen burn. This catalyst on-oil and regeneration cycle can be performed continuously, for instance, in a fluid bed regenerator system of the type shown schematically in FIG. 1, wherein a feed comprising reactants enter fluid bed reactor 11 via conduit 1 and effluent comprising product exits through conduit 5, and catalyst circulates between fluid bed reactor 11, apparatus 12, which strips hydrocarbons off the catalyst, and catalyst regenerator 13, via conduits 2, 3, and 4, respectively. Knowing when and how to regenerate the catalyst for improved performance while maximizing production of paraxylene is an area of intense research.

The present inventors have surprisingly discovered that under certain circumstances paraxylene selectivity is found to increase as the amount of coke on catalyst increases. Therefore, para-xylene selectivity and productivity can be maximized by controlling the desired coke level on catalyst while maintaining xylene yield at an acceptable value.

### SUMMARY OF THE INVENTION

The invention is directed to an improved process for the alkylation of aromatics hydrocarbons by contact of suitable reactants in the presence of suitable molecular sieve catalyst, and in preferred embodiments to an improved process for increasing the paraxylene selectivity of a zeolite catalyst suitable for the production of xylenes from benzene and/or toluene by alkylation with methanol.

In embodiments, the paraxylene selectivity and productivity is improved by controlling the amount of coke on the catalyst while maintaining xylene yield at an acceptable value.

In embodiments, the control of coke on the catalyst is achieved by attenuating process conditions in response to a change in the amount of coke on the catalyst. By way of example, this may include one or a combination of the following techniques: increasing catalyst on-oil time, decreasing catalyst residence time in the regenerator, reducing the air or oxygen supply to the regenerator, and decreasing catalyst circulation rate.

In embodiments the amount of coke on said catalyst is maintained within the range of about 0.5 wt % to about 5.0 wt %, or about 1.0 wt % to about 4.5 wt %, or about 1.5 wt % to about 4.0 wt %, or about 2.0 wt % to about 3.5 wt %, or about 2.5 wt % to about 3.0 wt %, with additional preferred ranges being from any lower limit to any upper limit just specified, thus including, by way of example, a range from about 2.5 wt % to about 5.0 wt %. The amount of coke on the catalyst will be understood to mean the average amount of coke on the bulk catalyst in the reactor, which from a practical matter can be taken to be represented by a sample taken, and analyzed for coke by any convenient means, such as thermogravimetic analysis.

In embodiments the catalyst is a molecular sieve catalyst which has already been selectivated, particularly by steam treatment, and in preferred embodiments is a phosphorus-containing molecular sieve, most preferably a phosphorus-containing molecular sieve comprising ZSM-5 which has been steam treated.

It is an object of the invention to improve at least one of selectivity, productivity, and yield of paraxylene in a process for the manufacture of xylenes by alkylation of benzene and/or toluene using a zeolite catalyst and methanol as the alkylation agent.

It is another object of the invention to provide a process which achieves a para-xylene selectivity of at least >90 wt % based on the amount of xylenes in the product stream.

These and other objects, features, and advantages will become apparent as reference is made to the following detailed description, preferred embodiments, examples, and appended claims.

### BRIEF DESCRIPTION OF THE DRAWINGS

In the accompanying drawings, like reference numerals are used to denote like parts throughout the several views.

FIG. 1 is a schematic of a reactor system including reactor and regenerator and some associated auxilliary devices and transfer piping.

FIG. 2 is a plot showing coke on catalyst impact on paraxylene selectivity for an embodiment of the invention.

FIG. 3 is a plot showing catalyst circulation rate impact on paraxylene selectivity for an embodiment of the invention.

FIG. 4 is a plot of the coke on catalyst impact on C11+ yield for an embodiment of the invention.

### DETAILED DESCRIPTION

The invention is directed to a process for alkylating aromatics in a fluid bed reactor by contact of the reactants with a zeolite catalyst and more particularly for increasing the paraxylene selectivity of a zeolite catalyst suitable for the production of xylenes from benzene and/or toluene by alkylation with methanol.

The invention also can be used, for example, in the para-selective production of other alkylaromatics, such as para-diethylbenzene and para-ethyltoluene.

According to an embodiment of the invention, there is a process in which the paraxylene selectivity is increased in a process in which toluene and/or benzene is/are alkylated with methanol to produce paraxylene in high selectivity (>90%), in the presence of a suitable molecular sieve catalyst, particularly a molecular sieve catalyst that has been steam treated, more particularly a phosphorus containing molecular sieve catalyst that has been stream treated, and preferably a catalyst comprising a phosphorus-containing ZSM-5 molecular sieve that has been stream treated.

One of the side reactions in the alkylation of benzene and/or toluene with methanol is the formation of coke through methanol reactions, aromatic reactions, and/or methanol-aromatic reactions, at least some of which is deposited on the catalyst. Without wishing to be bound by theory, the catalyst gradually loses its activity with time on-oil at least in part because of the accumulation of coke on the catalyst (and/or in the pores of the catalyst). As a result, the catalyst needs to be regenerated, typically under air, to remove coke after certain on-oil time.

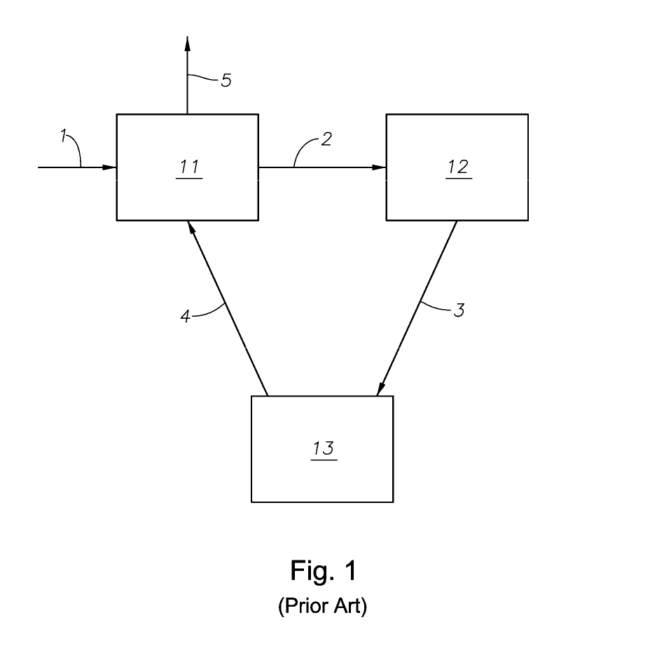

A fluid bed reactor system useful for the production of xylenes from toluene and/or benzene and methanol by contact of the reactants with a suitable zeolite catalyst can be one known in the prior art, such as illustrated schematically in the previously described FIG. 1. The reactor system, including each element shown in the figure, is known per se from Fluid Catalytic Cracking technology. The internals per se are well-known in the art and do not form an aspect of the present invention. One of ordinary skill in the art will recognize that details, such as valves, heaters, and the like, are not shown for convenience of view.

The present inventors have surprisingly discovered that paraxylene selectivity is found to increase as the amount of coke on catalyst increases. In embodiments the paraxylene selectivity and productivity is maximized by controlling the amount of coke on the catalyst while maintaining xylene yield at an acceptable value. The control of coke can be achieved, for instance, by one or a combination of the following techniques: increasing catalyst on-oil time, decreasing catalyst residence time in the regenerator, reducing the air or oxygen supply to the regenerator, and decreasing catalyst circulation rate, or a combination thereof.

In particular, as catalyst on-oil time increases, more coke is generated on the catalyst surface and/or in the catalyst pores, and thus the amount of coke on catalyst increases. Likewise, as catalyst residence time in the regenerator decreases, less coke is removed and the amount of coke on catalyst increases. Moreover; as catalyst recirculation rate decreases, less coked catalyst is regenerated and/or the extent of regeneration is decreased, and the amount of coke on catalyst increases.

An additional and also surprising benefit of increasing the amount of coke on catalyst in a process according to the present invention is to reduce the formation of heavy aromatics through the alkylation of toluene/xylene with olefins, which are byproducts of methanol reaction with itself. Making less of the heavy aromatics (C9+ aromatics) is beneficial because of their lower values compared to the xylenes and also because the purification of the desired paraxylene is made easier.

### Example 1

A pilot scale test was done using a fluidized bed reactor as discussed in U.S. Pat. No. 6,642,426, and fluid bed regenerator system of the type per se well-known in the art. The fluidized bed reactor was 10.2 cm (4 inches) in diameter and 8.2 m (27 feet) high. The regenerator was 15.2 cm (6 inches) in diameter and 25.4 cm (10 inches) high. The fluidized bed catalyst used contained about 4 wt % phosphorus and 10 wt % of 450/1

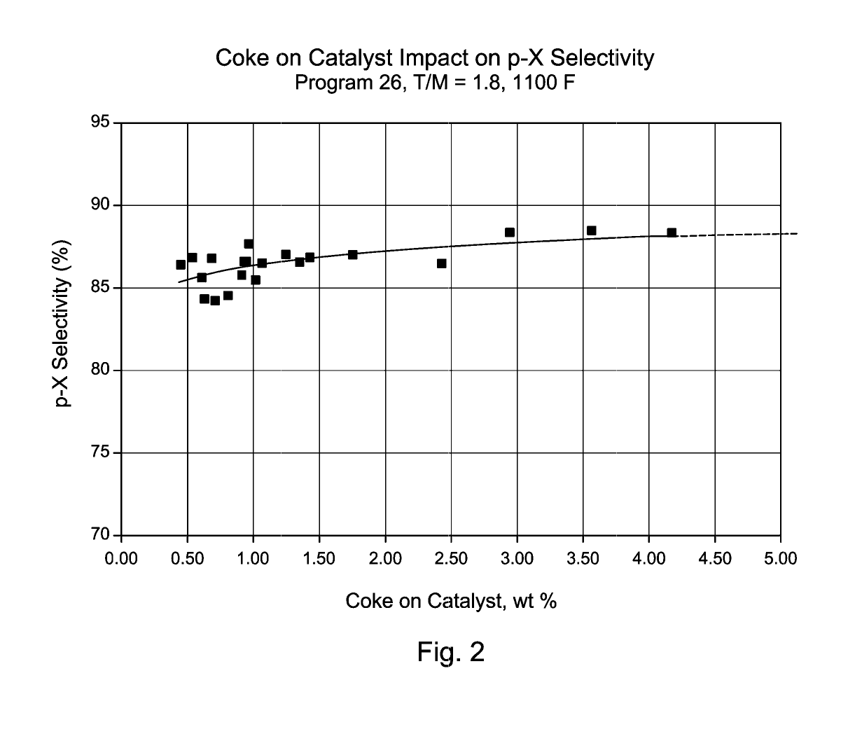

SiO₂/Al₂O₃ ZSM-5 zeolite in a binder comprising silica-alumina and clay. The catalyst was then steamed at about 1030° C. for about 45 minutes before being introduced to the reactor. The reactor and regenerator operated at 1100° F. (about 593° C.) and 20 psig (about 138 kPa). Water was cofed as well. The catalyst recirculation rate was about 104 lb/hr (47.2 kg/hr). Results are shown in FIG. 2, described below. The amount of coke on catalyst may be ascertained most conveniently by taking a sample of the catalyst after it has been stripped off hydrocarbons and prior to regeneration, for example by sampling along conduit 3 in FIG. 1 and then determining the amount of coke present by thermogravimetric analysis (TGA).

As shown in FIG. 2, product paraxylene selectivity increased from about 85% to about 88.5% as coke on the coke on catalyst increased from 0.5% to 4%. As indicated by the trend line in the plot, the benefit of para-xylene selectivity increase decreases as coke on the catalyst exceeds 4%.

### Example 2

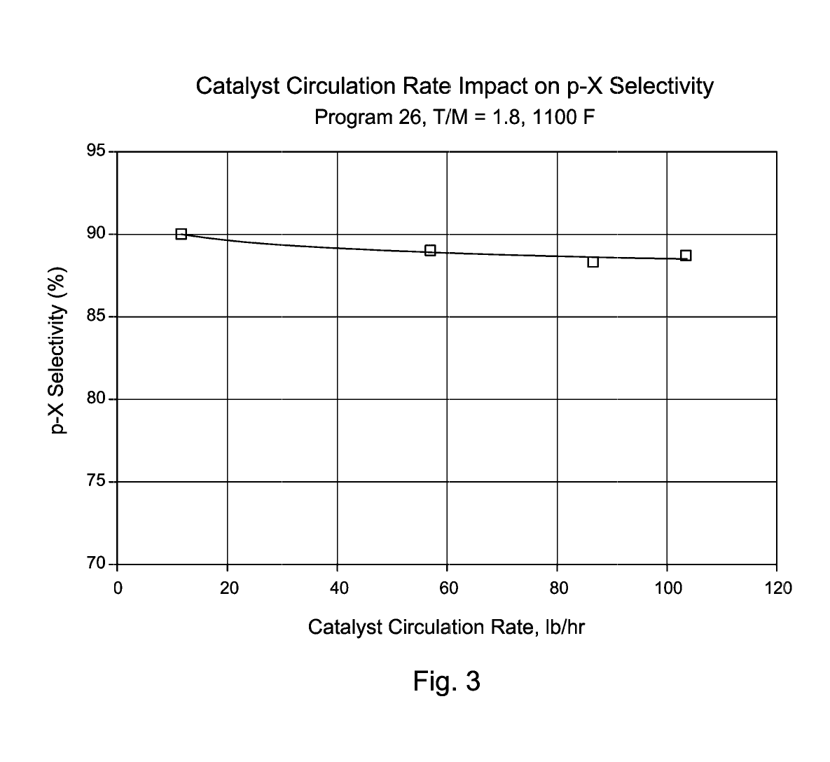

The reactor/regenerator in Example 1, above, was operated under the same initial conditions and the catalyst circulation rate was decreased over time from 104 lb/hr to 12 lb/hr (5.4 kg/hr). As shown in FIG. 3, paraxylene selectivity increases from 88 to 90%.

### Example 3

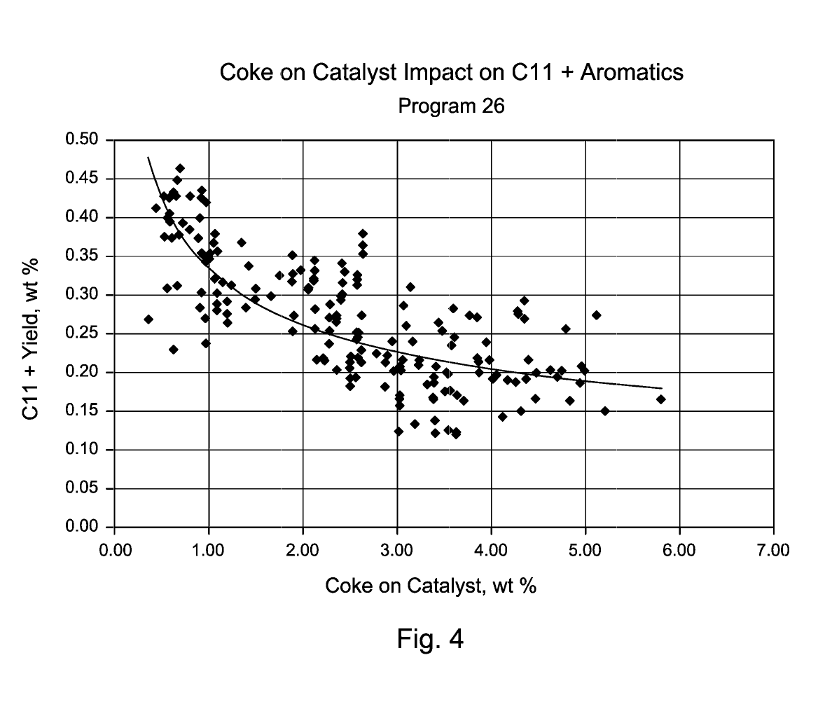

The same reactor/regenerator in the examples above was operated under the same initial conditions and the coke on catalyst increased from 0.5% to 5%. As shown in FIG. 4, the C11+ yield was analyzed over the same time frame and was shown to decrease from 0.45% to 0.2% (wt. % based on hydrocarbon in the effluent).

The alkylation process employed herein can employ any aromatic feedstock comprising toluene and/or benzene, although in general it is preferred that the aromatic feed contains at least 90 weight %, especially at least 99 weight %, of benzene, toluene or a mixture thereof. An aromatic feed containing at least 99 weight % toluene is particularly desirable.

Similarly, although the composition of the methanol-containing feed is not critical, it is generally desirable to employ feeds containing at least 90 weight %, especially at least 99 weight %, of methanol.

The catalyst employed in the present process may be any catalyst suitable for the conversion of benzene and/or toluene to xylenes with methanol. In preferred embodiments the catalyst comprises a porous crystalline material, typically having a Diffusion Parameter for 2,2 dimethylbutane of about 0.1-15 sec⁻¹ when measured at a temperature of 120° C. and a 2,2 dimethylbutane pressure of 60 torr (8 kPa).

As used herein, the Diffusion Parameter of a particular porous crystalline material is defined as D/r²×10⁶, wherein D is the diffusion coefficient (cm²/sec) and r is the crystal radius (cm). The required diffusion parameters can be derived from sorption measurements provided the assumption is made that the plane sheet model describes the diffusion process. Thus for a given sorbate loading Q, the value Q/Q₁₃, where Q₁₃ is the equilibrium sorbate loading, is mathematically related to (Dt/r²)^{1/2} where t is the time (sec) required to reach the sorbate loading Q. Graphical solutions for the plane sheet model are given by J. Crank in “The Mathematics of Diffusion”, Oxford University Press, Ely House, London, 1967.

The porous crystalline material is preferably a medium-pore size aluminosilicate zeolite. Medium pore zeolites are generally defined as those having a pore size of about 5 to about 7 Angstroms, such that the zeolite freely sorbs molecules such as n-hexane, 3-methylpentane, benzene and p-xylene. Another common definition for medium pore zeolites involves the Constraint Index test which is described in U.S. Pat. No. 4,016,218, which is incorporated herein by reference. In this case, medium pore zeolites have a Constraint Index of about 1-12, as measured on the zeolite alone without the introduction of oxide modifiers and prior to any steaming to adjust the diffusivity of the catalyst. In addition to the medium-pore size aluminosilicate zeolites, other medium pore acidic metallosilicates, such as silicoaluminophosphates (SAPOs), can be used in the present process.

Particular examples of suitable medium pore zeolites include ZSM-5, ZSM-11, ZSM-12, ZSM-22, ZSM-23, ZSM-35, and ZSM-48, with ZSM-5 and ZSM-11 being particularly preferred. In one embodiment, the zeolite employed in the process of the invention is ZSM-5 having a silica to alumina molar ratio of at least 250, as measured prior to any treatment of the zeolite to adjust its diffusivity.

Zeolite ZSM-5 and the conventional preparation thereof are described in U.S. Pat. No. 3,702,886. Zeolite ZSM-11 and the conventional preparation thereof are described in U.S. Pat. No. 3,709,979. Zeolite ZSM-12 and the conventional preparation thereof are described in U.S. Pat. No. 3,832,449. Zeolite ZSM-23 and the conventional preparation thereof are described U.S. Pat. No. 4,076,842. Zeolite ZSM-35 and the conventional preparation thereof are described in U.S. Pat. No. 4,016,245. ZSM-48 and the conventional preparation thereof is taught by U.S. Pat. No. 4,375,573. The entire disclosures of these U.S. patents are incorporated herein by reference.

The medium pore zeolites described above are preferred for the present process since the size and shape of their pores favor the production of p-xylene over the other xylene isomers. However, conventional forms of these zeolites have Diffusion Parameter values in excess of the 0.1-15 sec⁻¹ range desired for the present process. Nevertheless, the required diffusivity can be achieved by severely steaming the zeolite so as to effect a controlled reduction in the micropore volume of the catalyst to not less than 50%, and preferably 50-90%, of that of the unsteamed catalyst. Reduction in micropore volume is derived by measuring the n-hexane adsorption capacity of the zeolite, before and after steaming, at 90° C. and 75 torr n-hexane pressure.

Steaming of the porous crystalline material is effected at a temperature of at least about 950° C., preferably about 950 to about 1075° C., and most preferably about 1000 to about 1050° C. for about 10 minutes to about 10 hours, preferably from 30 minutes to 5 hours.

To effect the desired controlled reduction in diffusivity and micropore volume, it may be desirable to combine the porous crystalline material, prior to steaming, with at least one oxide modifier, preferably selected from oxides of the elements of Groups IIA, IIIA, IIIB, WA, VA, VB and VIA of the Periodic Table (IUPAC version). Most preferably, said at least one oxide modifier is selected from oxides of boron, magnesium, calcium, lanthanum and most preferably phosphorus. In some cases, it may be desirable to combine the porous crystalline material with more than one oxide modifier, for example a combination of phosphorus with calcium and/or magnesium, since in this way it may be possible to reduce the steaming severity needed to achieve a target diffusivity value. The total amount of oxide modifier present in the catalyst, as measured on an elemental basis, may be between about 0.05 and about 20 wt. %, and preferably is between about 0.1 and about 10 wt. %, based on the weight of the final catalyst.

Where the modifier includes phosphorus, incorporation of modifier in the catalyst of the invention is conveniently achieved by the methods described in U.S. Pat. Nos. 4,356,338, 5,110,776, 5,231,064 and 5,348,643, the entire disclosures of which are incorporated herein by reference. Treatment with phosphorus-containing compounds can readily be accomplished by contacting the porous crystalline material, either alone or in combination with a binder or matrix material, with a solution of an appropriate phosphorus compound, followed by drying and calcining to convert the phosphorus to its oxide form. Contact with the phosphorus-containing compound is generally conducted at a temperature of about 25° C. and about 125° C. for a time between about 15 minutes and about 20 hours. The concentration of the phosphorus in the contact mixture may be between about 0.01 and about 30 wt. %.

After contacting with the phosphorus-containing compound, the porous crystalline material may be dried and calcined to convert the phosphorus to an oxide form. Calcination can be carried out in an inert atmosphere or in the presence of oxygen, for example, in air at a temperature of about 150 to 750° C., preferably about 300 to 500° C., for at least 1 hour, preferably 3-5 hours.

Representative phosphorus-containing compounds which may be used to incorporate a phosphorus oxide modifier into the catalyst of the invention include derivatives of groups represented by PX₃, RPX₂, R₂PX, R₃P, X₃PO, (XO)₃PO, (XO)₃P, R₃P═O, R₃P═S, RPO₂, RPS₂, RP(O)(OX)₂, RP(S)(SX)₂, R₂P(O)OX, R₂P(S)SX, RP(OX)₂, RP(SX)₂, ROP(OX)₂, RSP(SX)₂, (RS)₂PSP(SR)₂, and (RO)₂POP(OR)₂, where R is an alkyl or aryl, such as phenyl radical, and X is hydrogen, R, or halide. These compounds include primary, RPH₂, secondary, R₂PH, and tertiary, R₃P, phosphines such as butyl phosphine, the tertiary phosphine oxides, R₃PO, such as tributyl phosphine oxide, the tertiary phosphine sulfides, R₃PS, the primary, RP(O)(OX)₂ and secondary, R₂P(O)OX, phosphonic acids. such as benzene phosphonic acid, the corresponding sulfur derivatives such as RP(S)(SX)₂ and R₂P(S)SX, the esters of the phosphonic acids, such as dialkyl phosphonate, (RO)₂P(O)H, dialkyl alkyl phosphonates, (RO)₂P(O)R, and alkyl dialkylphosphinates, (RO)P(O)R₂; phosphinous acids, R₂PDX, such as diethylphosphinous acid, primary, (RO)P(OX)₂, secondary, (RO)₂PDX, and tertiary, (RO)₃P, phosphites, and esters thereof such as the monopropyl ester, alkyl dialkylphosphinites, (RO)PR₂, and dialkyl alkyphosphinite, (RO)₂PR, esters. Corresponding sulfur derivatives may also be employed including (RS)₂P(S)H, (RS)₂P(S)R, (RS)P(S)R₂, R₂PSX, (RS)P(SX)₂, (RS)₂PSX, (RS)₃P, (RS)PR₂, and (RS)₂PR. Examples of phosphite esters include trimethylphosphite, triethylphosphite, diisopropylphosphite, butylphosphite, and pyrophosphites such as tetraethylpyrophosphite. The alkyl groups in the mentioned compounds preferably contain one to four carbon atoms.

Other suitable phosphorus-containing compounds include ammonium hydrogen phosphate, the phosphorus halides such as phosphorus trichloride, bromide, and iodide, alkyl phosphorodichloridites, (RO)PCl₂, dialkylphosphorochloridites, (RO)PCl, dialkylphosphinochloroidites, R₂PCl, alkyl alkylphosphonochloridates, (RO)(R)P(O)Cl, dialkyl phosphinochloridates, R₂P(O)Cl, and RP(O)Cl₂. Applicable corresponding sulfur derivatives include (RS)PCl₂, (RS)₂PCl, (RS)(R)P(S)Cl, and R₂P(S)Cl.

Particular phosphorus-containing compounds include ammonium phosphate, ammonium dihydrogen phosphate, diammonium hydrogen phosphate, diphenyl phosphine chloride, trimethylphosphite, phosphorus trichloride, phosphoric acid, phenyl phosphine oxychloride, trimethylphosphate, diphenyl phosphinous acid, diphenyl phosphinic acid, diethylchlorothiophosphate, methyl acid phosphate, and other alcohol-P₂O₅ reaction products.

Representative boron-containing compounds which may be used to incorporate a boron oxide modifier into the catalyst of the invention include boric acid, trimethylborate, boron oxide, boron sulfide, boron hydride, butylboron dimethoxide, butylboric acid, dimethylboric anhydride, hexamethylborazine, phenyl boric acid, triethylborane, diborane and triphenyl boron.

Representative magnesium-containing compounds include magnesium acetate, magnesium nitrate, magnesium benzoate, magnesium propionate, magnesium 2-ethylhexoate, magnesium carbonate, magnesium formate, magnesium oxylate, magnesium bromide, magnesium hydride, magnesium lactate, magnesium laurate, magnesium oleate, magnesium palmitate, magnesium salicylate, magnesium stearate and magnesium sulfide.

Representative calcium-containing compounds include calcium acetate, calcium acetylacetonate, calcium carbonate, calcium chloride, calcium methoxide, calcium naphthenate, calcium nitrate, calcium phosphate, calcium stearate and calcium sulfate.

Representative lanthanum-containing compounds include lanthanum acetate, lanthanum acetylacetonate, lanthanum carbonate, lanthanum chloride, lanthanum hydroxide, lanthanum nitrate, lanthanum phosphate and lanthanum sulfate.

The porous crystalline material employed in the present process may be combined with a variety of binder or matrix materials resistant to the temperatures and other conditions employed in the process. Such materials include active and inactive materials such as clays, silica and/or metal oxides such as alumina The latter may be either naturally occurring or in the form of gelatinous precipitates or gels including mixtures of silica and metal oxides. Use of a material which is active, tends to change the conversion and/or selectivity of the catalyst and hence is generally not preferred. Inactive materials suitably serve as diluents to control the amount of conversion in a given process so that products can be obtained economically and orderly without employing other means for controlling the rate of reaction. These materials may be incorporated into naturally occurring clays, e.g., bentonite and kaolin, to improve the crush strength of the catalyst under commercial operating conditions. Said materials, i.e., clays, oxides, etc., function as binders for the catalyst. It is desirable to provide a catalyst having good crush strength because in commercial use it is desirable to prevent the catalyst from breaking down into powder-like materials. These clay and/or oxide binders have been employed normally only for the purpose of improving the crush strength of the catalyst.

Naturally occurring clays which can be composited with the porous crystalline material include the montmorillonite and kaolin family, which families include the subbentonites, and the kaolins commonly known as Dixie, McNamee, Georgia and Florida clays or others in which the main mineral constituent is halloysite, kaolinite, dickite, nacrite, or anauxite. Such clays can be used in the raw state as originally mined or initially subjected to calcination, acid treatment or chemical modification.

In addition to the foregoing materials, the porous crystalline material can be composited with a porous matrix material such as silica-alumina, silica-magnesia, silica-zirconia, silica-thoria, silica-beryllia, silica-titania as well as ternary compositions such as silica-alumina-thoria, silica-alumina-zirconia silica-alumina-magnesia and silica-magnesia-zirconia.

The relative proportions of porous crystalline material and inorganic oxide matrix vary widely, with the content of the former ranging from about 1 to about 90% by weight and more usually, particularly when the composite is prepared in the form of beads, in the range of about 2 to about 80 wt. % of the composite.

In one embodiment, the binder material comprises silica or a kaolin day. Procedures for preparing silica-bound zeolites, such as ZSM-5, are described in U.S. Pat. Nos. 4,582,815; 5,053,374; and 5,182,242. A particular procedure for binding ZSM-5 with a silica binder involves an extrusion process.

In the present process, the methanol and aromatic feeds are contacted with the catalyst described above with the catalyst particles being disposed in one or more fluidized beds. Each of the methanol and aromatic feeds can be injected into the fluidized catalyst in a single stage. However, in a preferred embodiment, the methanol feed is injected in stages into the fluidized catalyst at one or more locations downstream from the location of the injection of the aromatic reactant into the fluidized catalyst. For example, the aromatic feed can be injected into a lower portion of a single vertical fluidized bed of catalyst, with the methanol being injected into the bed at a plurality of vertically spaced intermediate portions of the bed and the product being removed from the top of the bed. Alternatively, the catalyst can be disposed in a plurality of vertically spaced catalyst beds, with the aromatic feed being injected into a lower portion of the first fluidized bed and part of the methanol being injected into an intermediate portion of the first bed and part of the methanol being injected into or between adjacent downstream catalyst beds.

Irrespective of the disposition of the catalyst, as the reaction proceeds the catalyst gradually deactivates as a result of build-up of carbonaceous material, generally referred to as “coke” on the catalyst. Thus, a portion of the catalyst in the one or more fluidized bed is generally withdrawn, either on a continuous or a periodic basis, and fed to a separate regenerator. In the regenerator, the catalyst, again in the form of a fluidized bed, is contacted with an oxygen-containing gas, such as air, at a temperature between about 400 and about 700° C. so as to burn off the coke and regenerate the catalyst. The regenerated catalyst is continuously or periodically returned to the alkylation reactor, whereas the exhaust gas from the regenerator is scrubbed to remove entrained catalyst fines. The separated fines can be returned to the regenerator and/or purged to control the build-up of fines in the catalyst inventory.

The conditions employed in the alkylation stage of the present process are not narrowly constrained but, in the case of the methylation of toluene, generally include the following ranges: (a) temperature between about 500 and about 700° C., such as between about 500 and about 600° C.; (b) pressure of between about 1 atmosphere and about 1000 psig (between about 100 and about 7000 kPa), such as between about 10 psig and about 200 psig (between about 170 and about 1480 kPa); (c) moles toluene/moles methanol (in the reactor charge) of at least about 0.2, and preferably from about 0.2 to about 20; and (d) a weight hourly space velocity (“WHSV”) for total hydrocarbon feed to the reactor(s) of about 0.2 to about 1000, preferably about 0.5 to about 500 for the aromatic reactant, and about 0.01 to about 100 for the combined methanol reagent stage flows, based on total catalyst in the reactor(s).

Any trade names used herein are indicated by a ™ symbol or ® symbol, indicating that the names may be protected by certain trademark rights, e.g., they may be registered trademarks in various jurisdictions. All patents and patent applications, test procedures (such as ASTM methods, UL methods, and the like), and other documents cited herein are fully incorporated by reference to the extent such disclosure is not inconsistent with this invention and for all jurisdictions in which such incorporation is permitted. When numerical lower limits and numerical upper limits are listed herein, ranges from any lower limit to any upper limit are contemplated. While the illustrative embodiments of the invention have been described with particularity, it will be understood that various other modifications will be apparent to and can be readily made by those skilled in the art without departing from the spirit and scope of the invention.

What is claimed is:

1. In a process for the alkylation of benzene and/or toluene with methanol in the presence of a catalyst suitable for said alkylation and characterized as a porous crystalline material having a Diffusion Parameter for 2,2 dimethylbutane of 0.1-15 sec⁻¹ when measured at a temperature of 120° C. and a 2,2 dimethylbutane pressure of 60 torr (8 kPa), in an apparatus comprising a fluidized bed reactor and a regenerator, including a cycling of said catalyst between said reactor, wherein coke is deposited on said catalyst by contacting said benzene and/or toluene with methanol in the presence of said catalyst under conversion conditions for a predetermined on-oil cycle time, and said regenerator, wherein coke is removed from said catalyst under regeneration conditions for a predetermined residence time, the improvement comprising carrying out said process so as to maintain coke deposits on said catalyst in the range of greater than 0.5 wt % to no more than 5.0 wt %, based on the weight of said catalyst, and maintaining said contacting under conditions, including on-oil cycle time, catalyst residence time in said regenerator, and catalyst recirculation rate, so as to maintain the coke deposits on said catalyst within said range.

2. The process of claim 1, including regenerating or rejuvenating said catalyst by treatment under oxidative conditions, reductive conditions, treatment with steam, and combinations thereof.

3. The process according to claim 1, wherein said catalyst comprises a steam-treated, phosphorus-containing ZSM-5 molecular sieve.

4. The process according to claim 1, wherein said regenerating comprises contact with an atmosphere comprising molecular oxygen.

5. The process according to claim 1, further including a step of attenuating catalyst on-oil time in response to a change in the amount of coke deposits on said catalyst, as measured by analysis of a succession of coked catalyst samples taken between said fluidized bed reactor and said regenerator.

6. The process according to claim 1, further including a step of attenuating catalyst residence time in the regenerator in response to a change in the amount of coke deposits on said catalyst, as measured by analysis of a succession of coked catalyst samples taken between said fluidized bed reactor and said regenerator.

7. The process according to claim 1, further including a step of attenuating the supply of oxygen to the regenerator in response to a change in the amount of coke deposits on said catalyst, as measured by analysis of a succession of coked catalyst samples taken between said fluidized bed reactor and said regenerator.

8. The process according to claim 1, further including a step of attenuating the catalyst circulation rate in response to a change in the amount of coke deposits on said catalyst, as measured by analysis of a succession of coked catalyst samples taken between said fluidized bed reactor and said regenerator.

9. The process according to claim 1, wherein said range is from 2.0 wt % to 5.0 wt %.

10. The process according to claim 1, wherein said range is from 2.5 wt % to 5.0 wt %.

11. The process according to claim 1, including: (i) a step of determining that the amount of coke on said catalyst is above 5.0 wt % based on the weight of said catalyst; followed by (ii) at least one step selected from the group consisting of: (a) decreasing catalyst on-oil time; (b) increasing catalyst residence time in said regenerator; (c) increasing the supply of oxygen to said regenerator; and (d) increasing catalyst circulation rate.

12. The process according to claim 1, including: (i) a step of determining that the amount of coke on said catalyst is below 2.0 wt % based on the weight of said catalyst; followed by (ii) at least one step selected from the group consisting of: (a) increasing catalyst on-oil time; (b) decreasing catalyst residence time in said regenerator; (c) decreasing the supply of oxygen to said regenerator; and (d) decreasing catalyst circulation rate.
